(19) United States
(12) Patent Application Publication
(10) Pub. No.: US 2019/0350200 A1
(43) Pub. Date: Nov. 21, 2019

(54) SYNERGISTIC ANTIMICROBIAL COMPOSITIONS

(71) Applicant: JUBILANT LIFE SCIENCES LIMITED, Noida, Uttar Pradesh (IN)

(72) Inventors: Vineet SHARMA, Noida (IN); Rwittika DE, Noida (IN); Pradeep Kumar VERMA, Gajraula (IN); Ashutosh AGARWAL, Noida (IN)

(73) Assignee: JUBILANT LIFE SCIENCES LIMITED, Noida, Uttar Pradesh (IN)

(21) Appl. No.: 16/347,794

(22) PCT Filed: Nov. 6, 2017
(86) PCT No.: PCT/IB2017/056921

§ 371 Date: May 6, 2019

(30) Foreign Application Priority Data
Nov. 7, 2016 (IN) 201611037903

(51) Int. Cl. A01N 33/12; A61K 31/14; A01N 43/40; A01N 43/50; A61K 31/4164; A61K 31/4412; A61K 31/4418; A61K 31/4425; A61K 31/60; A61K 8/368; A61K 8/41; A61K 8/49; A61Q 17/00; A61Q 19/00; A61Q 5/00

(52) U.S. Cl. CPC A01N 33/12; A61Q 5/006; A01N 43/40; A01N 43/50; A61K 31/4164; A61K 31/4412; A61K 31/4418; A61K 31/4425; A61K 31/60; A61K 8/368; A61K 8/416; A61K 8/4926; A61K 8/4946; A61Q 17/005; A61Q 19/007; A61K 31/14

(57) ABSTRACT

The present invention relates to synergistic antimicrobial compositions comprising quaternary ammonium compound and antimicrobial active, process of preparing the same and their use. The compositions of the present invention possess activity at lower concentration of the actives and are environmentally benign.

### FIELD OF THE INVENTION

The present invention relates to synergistic antimicrobial compositions comprising quaternary ammonium compound and antimicrobial active, process of preparing the same and their use for imparting antimicrobial activity to a surface and/or formulation. The present invention relates to a method of imparting antimicrobial activity to a formulation by incorporating the synergistic antimicrobial compositions of the present invention in the said formulation. Further, the invention relates to a method of imparting antimicrobial activity to a surface by applying the synergistic antimicrobial compositions of the present invention to the said surface. Also, the present invention relates to an environmentally benign synergistic antimicrobial composition comprising quaternary ammonium compound and antimicrobial active, wherein the actives possess activity at lower concentration of the actives.

### BACKGROUND OF THE INVENTION

Microbes such as bacteria, fungus, yeasts and mould are most common cause of any infection. They affect any surface which provides favourable temperature, moisture, oxygen and pH for their growth. In humans, the most common conditions arising out of such infections are dandruff, athlete's foot, jock itch, ringworm, plaque, pruritis, gingivitis and yeast infections. With rising health and hygiene awareness, the market offers a wide range of personal care, OTC, household and industrial use products containing one or more antimicrobial compound that can help in combating these microbes.

Quaternary ammonium compounds, owing to their surfactant and antimicrobial properties, find widespread applications in the field of cosmetics, food, personal care, medicine, pharmaceuticals, water disinfection, leather, textile, paint and coating industry etc. The most commonly used quaternary ammonium compounds are benzalkonium chloride, dodecyl dimethyl ammonium chloride, alkyl benzyl dimethyl ammonium chloride, benzyl-C₈-₁₈-alkyl dimethyl ammonium chloride, dodecyl benzyl dimethyl ammonium bromide and dodecyl dimethyl ammonium bromide. Quaternary ammonium compounds like benzalkonium chloride, benzethonium chloride and cetrimonium bromide are also approved as preservatives for use in cosmetics.

There are a lot of prior arts available related to the diverse applications of quaternary ammonium compounds.

U.S. patent publication No. 2006/0241190 discloses a skin treatment composition for treatment of psoriasis or eczema comprising quaternary ammonium compounds as keratolytic agents. The composition in addition to quaternary ammonium compounds comprises vanilla exact, ammonium chloride and potassium chloride. Quaternary ammonium compounds that have been disclosed as keratolytic agents are benzalkonium chloride, benzethonium chloride, cetalkonium chloride, cetrimide, cetrimonium bromide, cetylpyridinium chloride, glycidyl trimethyl and stearalkonium chloride.

Eley, in British Dental Journal 1999, 186, 286-296, reported the plaque inhibitory activity of cetylpyridinium chloride.

U.S. patent publication No. 2008/0057015 discloses hair care compositions comprising cetylpyridinium chloride as hair growth inhibiting agent. The composition is suitable for longer lasting hair style appearance, including coloration and grooming on the head, neck, and face of consumers.

U.S. patent publication No. 2016/0066571 discloses disinfectant compositions comprising quaternary ammonium compounds viz. didecyl dimethyl ammonium chloride and/or C₈-C₁₈ alkyldimethylbenzylammonium chloride and hydrogen peroxide having enhanced antimicrobial activity and effective against microorganisms such as Staphylococcus, Pseudomonas, Bacillus, Hepatitis, Rotavirus, Rhinovirus and Mycobacterium terrae.

U.S. patent publication 2012/0177712 discloses bipolar antimicrobial particle useful in personal care, fabrics and textile care composition. The bipolar particle comprises clay with quaternary ammonium compounds such as cetylpyridinium chloride, cetyltrimethylammonium chloride, cetyltrimethylammonium bromide, benzalkonium chloride, benzethonium chloride, cetrimide or quaternium.

Use of quaternary ammonium compounds as surfactant in personal care is well known and has been disclosed in many prior arts.

U.S. publication Nos. 2011/0003016 and 2012/0064136 disclose the use of quaternary ammonium compounds such as cetylpyridinium chloride as cationic surfactant in a hair treatment and anti-aging compositions respectively.

PCT publication No. WO98/023258 discloses antimicrobial personal care compositions comprising piroctone olamine as active, polyethylenimine as polymer and a surfactant. The surfactant can be selected from anionic, nonionic, amphoteric, zwitterionic or cationic surfactants or their mixtures. Cetylpyridinium chloride has been disclosed as one of the cationic surfactant.

U.S. Pat. No. 8,501,743 discloses a eutectic mixture of azole based antidandruff agent and menthol in combination with surfactant as hair/scalp care composition. It discloses that eutectic mixtures can be used to enhance deposition of benefit agents. It discloses the use of quaternary ammonium compounds as cationic conditioning polymers in these compositions. Some of the quaternary ammonium compounds disclosed are cetylpyridinium chloride, octyltrimethyl ammonium chloride, cetyltrimethyl ammonium chloride, dodecyldimethyl ammonium chloride and the like.

U.S. Pat. No. 7,871,649 discloses antimicrobial compositions of benzalkonium chloride or benzethonium chloride with essential oils. The compositions are effective against Staphylococcus, Escherichia and Salmonella species.

PCT publication No. WO2015/033351 discloses that antimicrobial combination of zinc pyrithione and C₈-₁₈ quaternary ammonium compounds show enhanced antimalassizia activity at lower concentration of the actives.

PCT publication No. WO2016/018718 discloses a synergistic combination for reducing, or inhibiting, or preventing microbial growth. The composition claimed is of tris(hydroxymethyl) nitromethane (THNM) with quaternary ammonium compounds viz. N-alkyl dimethylbenzyl ammonium chloride, N-alkyldimethylbenzyl ammonium chloride, didecyl ammonium chloride, benzalkonium chloride or polyquat 60.

Although antimicrobial combinations are known in the art, there is a need of additional antimicrobial combinations which can provide broad spectrum activity at lower concentration of the actives. The problem addressed by the present invention is to provide such combinations.

### SUMMARY OF THE INVENTION

The main objective of the present invention is to provide synergistic antimicrobial compositions comprising quaternary ammonium compound and antimicrobial active.

In one embodiment, the present invention provides antimicrobial compositions comprising quaternary ammonium compound and antimicrobial active, said antimicrobial composition having higher antimicrobial activity as compared to the combined individual antimicrobial activity of the quaternary ammonium compound and the antimicrobial active, against a wide range of microorganisms.

In one embodiment the present invention provides synergistic antimicrobial compositions comprising quaternary ammonium compound and antimicrobial active, wherein the quaternary ammonium compound is present in an amount of 0.0025- 50% w/w and antimicrobial active is present in an amount of 0.0025-25% w/w.

In another embodiment of the present invention, the antimicrobial active is selected from the group comprising antifungal, antibacterial, anti-viral, anti-algal, anti-yeast and mold and anti-parasitic agent.

In one embodiment, the present invention provides synergistic antimicrobial composition comprising cetylpyridinium chloride and an antimicrobial active selected from climbazole, ketoconazole, ciclopirox olamine, octopirox or salicylic acid and combinations thereof.

In another embodiment, the present invention provides process of preparing synergistic antimicrobial compositions comprising quaternary ammonium compound and an antimicrobial active.

In another embodiment, the present invention provides the use of synergistic antimicrobial composition comprising quaternary ammonium compound and antimicrobial active for imparting antimicrobial activity to a surface to which they are applied.

In yet another embodiment, the present invention relates to a method of imparting antimicrobial activity to a formulation by incorporating the synergistic antimicrobial compositions of the present invention in the said formulation.

In yet another embodiment, the present invention provides a method of preventing or inhibiting microbial growth on a surface by applying a synergistic antimicrobial composition comprising quaternary ammonium compound and an antimicrobial active to the said surface.

In one embodiment, the synergistic antimicrobial compositions of the present invention are suitable for use in and as antidandruff, anti-acne, anti-wart, anti-fungal, anti-eczema, anti-psoriasis, anti-athlete's foot, anti-ringworm, anti-pruritic, anti-candidiasis, anti-crack, anti-dermatitis, anti-tinea, anti-vitiligo, would healing and dry skin formulations.

In one embodiment, the present invention relates to the use of synergistic antimicrobial composition comprising quaternary ammonium compound and antimicrobial active in personal care, cosmetic, pharmaceutical, home care, hospital disinfectants, surface disinfectant, laundry care and/or industrial products.

### DETAILED DESCRIPTION OF THE INVENTION

The present invention relates to synergistic antimicrobial compositions comprising quaternary ammonium compound and an antimicrobial active, process of preparing the same and their use for imparting antimicrobial activity to a surface or a formulation by applying it to the said surface and/or by incorporating it in the said formulation. The present invention relates to a method of imparting antimicrobial activity to a formulation by incorporating the synergistic antimicrobial compositions of the present invention in the said formulation. Further, the invention relates to a method of imparting antimicrobial activity to a surface by applying the synergistic antimicrobial compositions of the present invention to the said surface.

The inventors of the present invention have found that the combination of quaternary ammonium compound and certain antimicrobial active showed effective synergistic antimicrobial activity at lower concentrations of actives relative to their individual antimicrobial activities combined together, against a wide range of microorganisms. These antimicrobial compositions possess the desired antimicrobial activity at lower concentration of the actives as compared to when used alone.

One would appreciate that the aesthetic appeal of consumer product formulations have significant effects on consumer acceptance and usage. Transparent formulations provide greater sensorial benefits and consumer acceptability. However, transparent formulations of actives with limited water solubility or water insoluble actives cannot be readily developed. Because of limited solubility they may be required to be incorporated in higher amount to ensure the delivery of the desired concentration at the active site. The present invention overcomes this limitation by providing synergistic combinations where the actives are effective at lower concentration and at the same time the composition provides a system which can solubilize water insoluble actives or actives with limited water solubility. This makes the composition stable to be incorporated into various transparent formulations.

The invention is described herein in detail using the terms defined below unless otherwise specified.

The term “microorganism” as used herein refers to fungi, bacteria, algae, yeast, mold and virus.

The term “antimicrobial active” refers to a compound capable of inhibiting the growth or killing microorganisms such as fungi, bacteria, algae, yeast, mold and virus.

The term “synergistic” as used herein refers to the effect where the antimicrobial activity of the combination of two compounds is more than the addition of the antimicrobial activity of the two compounds when tested alone.

The term “personal care formulation” as used herein refers to various toiletries and cosmetic preparation used for general health, hygiene and grooming.

The term “industrial use formulations” refers to metalworking fluids, fuels, paints, coatings, adhesives, sealants, elastomers, swimming pool products, wood products, plastic products, woven or nonwoven fibers, and the likes.

The term “laundry care formulations” as used herein refers to products formulated to remove dirt from clothes. Additionally it also removes odour and provide conditioning to the fabric. The formulations can be used for manual washing or machine washing.

The term “home care formulations” as used herein refers to products formulated for cleaning, disinfecting, rinsing or care of dishes, utensils, cars, floors, tiles, ceramics, carpets, rugs, mats and the likes. The formulations can be used for manual washing or machine washing.

The term “pharmaceutical formulation” as used herein refers to both prescription and non-prescription or over the counter (OTC) products.

The term “quaternary ammonium compound (QAC)” as used herein refers to salts of quaternary ammonium cations with anions. The quaternary ammonium cations are positively charged ions in which a central nitrogen atom is attached to same or different four straight chain or branched alkyl group or in which a central nitrogen atom is a part of pyridine ring and is attached to alkyl group.

The term “surfactant” as used herein refers to substances which lower the surface tension of the medium in which it is dissolved, and/or the interfacial tension with other phases, and, accordingly, is positively adsorbed at the liquid/vapour and/or at other interfaces. Surfactants have a hydrophobic part and a hydrophilic part. The hydrophobic part consists of an uncharged carbohydrate group that can be straight, branched, cyclic or aromatic. Depending on the nature of the hydrophilic part the surfactants are classified as anionic, cationic, non-ionic, or amphoteric.

The term “anionic surfactant” as used herein refers to those surfactants where the hydrophilic part consists of a negatively charged group.

The term “cationic surfactant” as used herein refers to those surfactants where the hydrophilic part consists of a positively charged group.

The term “non-ionic surfactant” as used herein refers those surfactants where the hydrophilic part is not charged.

The term “amphoteric surfactant” as used herein refers to those surfactants wherein hydrophilic part can be either positively or negatively charges depending on the pH of the solution. They can act as anionic surfactant in an alkaline solution or as cationic surfactant in an acidic solution.

The term “surfactant or surfactant system” as used herein refers to one or more surfactants selected form anionic surfactant, cationic surfactant, non-ionic surfactant, amphoteric surfactants or a combination thereof.

The term “suitable solvent” as used herein refers to any liquid or mixture of liquids which aids in dissolving or diluting any other substance or substance mixture or a product.

The term “rheology modifier” as used herein refers to compounds/polymers which alter the thickness or viscosity of the system.

The term “suspending agent”, are as used herein refers agents which help to reduce the sedimentation rate of particles in suspension.

The term “dispersant” as used herein are substances which facilitate the dispersion of aggregates and improve the kinetic stability of the particles.

The abbreviation “AA” refers to antimicrobial active and “QAC” refers to quaternary ammonium compound.

In one embodiment, the present invention provides synergistic antimicrobial compositions comprising quaternary ammonium compound and an antimicrobial active.

In another embodiment, the present invention provides synergistic antimicrobial compositions comprising quaternary ammonium compound and an antimicrobial active, said antimicrobial composition having higher antimicrobial activity as compared to the combined individual antimicrobial activity of the quaternary ammonium compound and the antimicrobial active, against a wide range of microorganisms.

In another embodiment of the present invention, the antimicrobial active is selected from the group comprising antifungal, antibacterial, anti-viral, anti-algal, anti-parasitic, and anti-yeast and mold compounds.

The quaternary ammonium compound of the present invention is selected from the group comprising methyltrioctyl ammonium halides, cetyltrimethyl ammonium halides, decyltrimethyl ammonium halides, didecyldimethyl ammonium halides, trimethyltetradecyl ammonium halides, methyl pyridinium halides, ethyl pyridinium halides, cetrimonium halides, dodecyl (lauryl) pyridinium halides, tetradecyl (myristyl) pyridinium halides, hexadecyl (cetyl) pyridinium halides, octadecyl(stearyl) pyridinium halides, alkylbenzyldimethyl ammonium halides, benzalkonium halides or benzalkonium saccharinates with alkyl chain lengths of C₈-C₁₈ and combinations thereof.

In one embodiment, the quaternary ammonium salt of the present invention is selected from methyltrioctyl ammonium chloride, methyltrioctyl ammonium bromide, cetyl trimethyl ammonium chloride, cetyl trimethyl ammonium bromide, decyltrimethyl ammonium chloride, dodecyl (lauryl) pyridinium chloride, tetradecyl (myristyl) pyridinium chloride, cetrimonium chloride, cetrimonium bromide, benzalkonium chloride, benzalkonium bromide, benzalkonium saccharinate, cetylpyridinium chloride, cetylpyridinium bromide and octadecylpyridinium chloride and combinations thereof.

The antimicrobial active of the present invention is selected from the group comprising abafungin, albaconazole, bifonazole, butoconazole, clotrimazole, climbazole, econazole, efinaconazole, epoxiconazole, fluconazole, fenticonazole, isavuconazole, itraconazole, isoconazole, ketoconazole, luliconazole, miconazole, omoconazole, oxiconazole, posaconazole, propiconazole, ravuconazole, sertaconazole, sulconazole, spectrazole, tioconazole, terconazole, tolyltriazole, voriconazole, ciclopirox olamine (ciclopirox), octopirox (piroctone olamine), selenium sulfide, sulfur, coal tar, salicylic acid, benzotriazole, 2-mercaptobenzothiazole (MBT), lactic acid, pyruvic acid, urea, benzoyl peroxide, N-acetylcysteine, retinoids, tretinoin, retinoic acid, retinol and retinol palmitate, isotretinoin-13-cis-retinoic acid, tetracycline, erythromycin, minocycline, clindamycin, tolnaftate, terbinafine, methyl-isothiazolinone, chloromethyl-isothiazolinone, benz-isothiazolinone, octyl-isothiazolinone, dichlorooctyl-isothiazolinone, butylbenz-isothiazolinone, iodopropynyl butylcarbamate and combinations thereof.

In one embodiment, the antimicrobial active is selected from the group comprising, clotrimazole, climbazole, fluconazole, ketoconazole, ciclopirox olamine (ciclopirox), octopirox, selenium sulfide, sulfur, salicylic acid and combinations thereof.

In one embodiment the present invention provides synergistic antimicrobial composition comprising cetylpyridinium chloride and an antimicrobial active selected from climbazole, ketoconazole, ciclopirox olamine, octopirox or salicylic acid.

In one embodiment, the present invention provides synergistic antimicrobial compositions comprising quaternary ammonium compound and antimicrobial active, wherein the quaternary ammonium compound is present in an amount of 0.0025- 50% w/w and antimicrobial active is present in an amount of 0.0025-25% w/w.

In another embodiment, the present invention provides synergistic antimicrobial compositions comprising quaternary ammonium compound and antimicrobial active, wherein the quaternary ammonium compound is present in an amount of 5-25% w/w and antimicrobial active is present in an amount of 2.5-15% w/w.

In yet another embodiment, the present invention provides synergistic antimicrobial compositions comprising quaternary ammonium compound and antimicrobial active, wherein the quaternary ammonium compound is present in an amount of 2-40% w/w and antimicrobial active is present in an amount of 1-20% w/w.

In one embodiment, the present invention provides synergistic antimicrobial compositions wherein the quaternary ammonium compound and antimicrobial active are present in a ratio of 1:10 to 10:1.

In one embodiment, the present invention provides synergistic antimicrobial compositions wherein the quaternary ammonium compound and antimicrobial active are present in a ratio of 1:5 to 5:1.

In one embodiment, the present invention provides synergistic antimicrobial composition comprising quaternary ammonium compound and antimicrobial active wherein the said antimicrobial composition can be transparent or opaque.

In one embodiment, the present invention provides a process for preparing opaque antimicrobial compositions comprising quaternary ammonium compound and antimicrobial active, said process comprising:

(a) mixing the antimicrobial active with surfactant and mixing the same with aqueous mixture of dispersant to obtain a slurry,

(b) preparing a solution of the quaternary ammonium compound in suitable solvent,

(c) mixing slurry obtained in step (a) and solution of step (b),

(d) adding aqueous mixture of rheology modifier, pH regulator, preservative optionally.

In another embodiment, the present invention provides a process for preparing a transparent anti-microbial composition comprising a quaternary ammonium compound and antimicrobial active, said process comprising:

(a) preparing a solution of antimicrobial active in suitable solvent,

(b) preparing a solution of quaternary ammonium compound in suitable solvent,

(c) mixing quaternary ammonium compound solution and antimicrobial active solution,

(d) adding aqueous mixture of rheology modifier, pH regulator, preservative optionally.

The suitable solvent used in the process of the present invention is selected from the group comprising propylene glycol, glycerol, sorbitol, PEG 400, polyglycol 500 DME, ethylene glycol monoethyl ether, diethylene glycol monomethyl ether, diethylene glycol monoethyl ether, diethylene glycol dimethyl ether (diglyme), triethylene glycol dimethyl ether (triglyme), tetraethylene glycol dimethyl ether (tetraglyme) and combinations thereof; water, phenoxyethanol, lactams such as 2-pyrrolidone, N-methyl pyrrolidone (NMP), polyvinylpyrrolidone (PVP) and combinations thereof; surfactants such as anionic, cationic, non-ionic, amphoteric surfactants and mixtures thereof.

The anionic surfactant used in the process of the present invention is selected from the group comprising sodium, potassium or ammonium salts of long chain sulphates having carbon chain lengths 6-14, preferably sodium lauryl sulfate (SLS), sodium laureth sulfate (SLES), triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, ammonium lauryl sulfate, ammonium laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, monoethanolamine lauryl sulfate, sodium tridecyl benzene sulfonate, sodium dodecyl benzene sulfonate, sodium cocoyl isethionate, amino acid derived surfactants and combinations thereof.

The cationic surfactant used in the process of the present invention is selected from the group comprising cetyl pyridinium chloride, stearyl pyridinium chloride, methyl or ethyl cetyl pyridinium chloride, aralkyl ammonium halides such as benzyl triethyl ammonium chloride, benzalkonium chloride, cetalkonium chloride, benzethonium chloride, lauryltrimethyl ammonium halide, cetrimonium halide or cetyltrimethyl ammonium halide, glycidyltrimethylammonium halide, tallowtrimethyl ammonium chloride, cocotrimethyl ammonium chloride, vitamin B6 hydrochloride, behenyltrimethyl ammonium chloride (BTAC), octyltrimethyl ammonium chloride, octyldimethylbenzyl ammonium chloride, decyldimethylbenzyl ammonium chloride, stearyldimethylbenzyl ammonium chloride, didodecyldimethyl ammonium chloride, dioctadecyldimethyl ammonium chloride, dipalmitoylethyldimethyl ammonium chloride and combinations thereof.

The non-ionic surfactant used in the process of the present invention is selected from the group comprising Lamesoft PO65, polyoxyethylene (20) sorbitan monooleate (Tween 80), polyoxyethylene (20) sorbitan monolaurate (Tween 20), ethoxylated sorbitan monolaurate (Crillet 180) and combinations thereof.

The amphoteric surfactant used in the process of the present invention is selected from the group comprising cocamidopropyl betaine (CAPB) or cocamide DEA and combinations thereof.

The dispersants and/or rheology modifier and/or suspending agent used in the process of the present invention is selected from the group comprising synthetic silicates, castor oil based thixotropes and organic thixoptropes, carboxymethylcellulose, organoclays, synthetic clays, polymers of acrylic acid cross-linked with polyalkenyl ethers or divinyl glycol, Stepan TAB-2, Stepan SAB-2, Carbopol ETD 2020, Carbopol Aqua SF-1, Carbopol Ultrez 20, Rheocare TTA, Rheocare C Plus, xanthum gum, dehydroxanthan gum like Amaze XT, methyl hydroxyethylcellulose like Structure Cell 12000 and combinations thereof. Synthetic silicates are selected from but are not limited to sodium aluminium silicate, magnesium aluminum silicates and the likes; organoclays such as Claytone, Tixogel and the likes; synthetic clay such as Veegum, Laponite and the likes.

The antimicrobial compositions of the present invention are stable in wide pH range. Owing to such broad range of pH stability, they are suitable for being incorporated into formulations with diverse application.

The synergistic antimicrobial compositions of the present invention are environmentally benign as the actives show the desired activity at very low concentrations thereby reducing the overall toxicological impact on the environment.

The synergistic antimicrobial compositions of the present invention can directly be incorporated into personal care, cosmetic, pharmaceutical, laundry care or industrial formulations in aqueous medium in the required concentration of actives without any difficulty of stability or precipitation. These formulations can be either transparent or opaque.

In one embodiment, the antimicrobial composition of the present invention can be incorporated into a transparent personal care, cosmetic, pharmaceutical, laundry care, home care or industrial formulation without any difficulty of stability or precipitation.

The personal care, cosmetic, pharmaceutical, laundry care, home care or industrial formulations are present in the form of emulsion, suspension, cream, solution, lotion, gel, serum, spray, mousse, cake and powder.

The personal care and cosmetic formulations can be for “rinse off” or “leave on” applications and are selected from soap, shampoos, shower gel, conditioners, hair gel, wipes, moisturizers, cream, sunscreens, perfumes, deodorizers, antiperspirants, toothpaste, creams and gels, mouthwashes.

Examples of pharmaceutical products in which the composition of the present invention can be incorporated include both OTC and prescription products.

Examples of industrial use formulations in which the composition of the present invention can be incorporated include metalworking fluids, fuels, paints, coatings, adhesives, sealants, elastomers, swimming pool products, wood products, wood preservatives plastic products, woven or nonwoven fibres, corrosion inhibitors, preservatives and the likes.

Examples of laundry care formulations in which the composition of the present invention can be incorporated include fabric softener, fabric freshener sprays, cleaning detergents, liquid all-purpose cleaner, and fabric conditioners.

Examples of home care formulations in which the composition of the present invention can be incorporated include floor cleaners, disinfectants, dish-washing liquids, car washes, tile cleaners, carpets and rugs cleaners and the likes.

In one embodiment, the synergistic antimicrobial compositions of the present invention are suitable for use in and as antidandruff, anti-acne, anti-wart, anti-fungal, anti-eczema, anti-psoriasis, anti-athlete's foot, anti-ringworm, anti-pruritic, anti-candidiasis, anti-crack, anti-dermatitis, anti-tinea, anti-vitiligo, would healing and dry skin formulations.

In another embodiment, the synergistic antimicrobial compositions of the present invention are suitable for use in and as antidandruff formulations.

In another embodiment, the antimicrobial compositions of the present invention are suitable for being incorporated into anti-malassizia and antifungal formulations.

In another embodiment, the antimicrobial compositions of the present invention are suitable for being incorporated into antibacterial formulations.

In one embodiment, the antimicrobial compositions of the present invention are suitable for use as preservative in various personal care, cosmetic, pharmaceutical, laundry care, home care and/or industrial formulations.

In one embodiment, the present invention relates to the use of synergistic antimicrobial composition comprising quaternary ammonium compound and antimicrobial active in personal care, cosmetic, pharmaceutical, home care, laundry care and/or industrial products.

In another embodiment, the present invention provides the use of synergistic antimicrobial composition comprising quaternary ammonium compound and antimicrobial active for imparting antimicrobial activity to a surface to which they are applied.

In yet another embodiment, the present invention relates to a method of imparting antimicrobial activity to a formulation by incorporating the synergistic antimicrobial compositions in the said formulation. Example of formulations into which the composition of the present invention can be incorporated include but are not limited to personal care, cosmetic, pharmaceutical, home care, hospital disinfectants, laundry care and/or industrial formulations.

In yet another embodiment, the present invention provides a method of imparting antimicrobial activity to a surface by applying synergistic antimicrobial compositions comprising quaternary ammonium compound and an antimicrobial active to the said surface. The composition of the present invention provide antimicrobial effect to the surface they are applied. Example of surfaces to which the composition of the present invention can be applied include but is not limited to skin, scalp, nails and teeth of humans and animals, metallic and non-metallic substrates, woven and non-woven fabrics, polymers, plastics, paper, wooden surfaces and ceramics.

The synergistic antimicrobial compositions of the present invention are effective against a wide variety of microorganisms. Examples of microorganisms that are effectively inhibited or killed by the composition of the invention include but are not limited to Aspergillus niger, Alcaligenes faecalis, Aureobasidium pullulans, Acremonium butryi, Bacillus cereus, Cephalosporium, Candida sp, Candida albicans, Chlorella spp, Chlorella vulgaris, Chaetomium globosum, Citrobacter freundii, Escherichia spp, Escherichia coli, Fusarim spp, Klebsiella pneumonia spp., Listeria spp., Malassezia spp, Malassezia furfur, Malassezia sympodialis, Malassezia globosa, Mycobacterium chelonae, Oscillitoria spp, Penicillium citrinum, Propionibacterium acne, Proteus mirabilis, Pseudomonas aeruginosa, Pseudomonas fluorescens, Pityrosporum ovale, Pseudomonas oleovorans, Pseudomonas rubescens, Pseudomonas stutzeri, Staphylococcus aureus, Salmonella enteric, Shewanella putrefaciens, Streptococcus pyogenes, Staphylococcus epidermidis, Trichophyton mentagrophytes, Trichophyton rubrum.

In one embodiment, the present invention relates to a method of inhibiting the growth of microorganisms selected from the group comprising Aspergillus niger, Alcaligenes faecalis, Aureobasidium pullulans, Acremonium butryi, Bacillus cereus, Cephalosporium, Candida sp, Candida albicans, Chlorella spp, Chlorella vulgaris, Chaetomium globosum, Citrobacter freundii, Escherichia spp, Escherichia coli, Fusarim spp, Klebsiella pneumonia spp., Listeria spp., Malassezia spp, Malassezia furfur, Malassezia sympodialis, Malassezia globosa, Mycobacterium chelonae, Oscillitoria spp, Penicillium citrinum, Propionibacterium acne, Proteus mirabilis, Pseudomonas aeruginosa, Pseudomonas fluorescens, Pityrosporum ovale, Pseudomonas oleovorans, Pseudomonas rubescens, Pseudomonas stutzeri, Staphylococcus aureus, Salmonella enteric, Shewanella putrefaciens, Streptococcus pyogenes, Staphylococcus epidermidis, Trichophyton mentagrophytes, Trichophyton rubrum using quaternary ammonium compound.

In one embodiment, the present invention also provides the use of quaternary ammonium compound for inhibiting the growth of Malassezia spp.

In one of the embodiment, the present invention provides the use of cetylpyridinium chloride in an amount of 0.05-10% for inhibiting the growth of Malassezia spp.

The present invention also provides process of preparing the various personal care, cosmetic, pharmaceutical, laundry care, home care or industrial formulations comprising the synergistic antimicrobial composition of the present invention.

The present invention provides a process for preparing anti-microbial formulations comprising the synergistic antimicrobial composition comprising quaternary ammonium compound and an antimicrobial active.

The personal care, cosmetic, pharmaceutical, laundry care, home care or industrial formulations can be prepared by any of their respective conventional methods of preparations.

### EXAMPLES

The invention is explained in detail in the following examples which are given solely for the purpose of illustration only and therefore should not be construed to limit the scope of the invention.

### Example 1:

Preparation of anti-microbial compositions: The antimicrobial actives were dissolved in a suitable solvent and/or surfactant mixture. Cetyl pyridinium chloride (CPC) solution was prepared by dissolving in PG-water mixture. The antimicrobial active solution and CPC solution were mixed slowly with continuous mixing. A stable colourless solution was obtained.

TABLE 1
Various compositions of antimicrobial active and CPC

| Ingredients | Comp-1 | Comp -2 | Comp -3 | Comp -4 | Comp -4 | Comp -5 |
|---|---|---|---|---|---|---|
| CPC | 30.0% | 15.0% | 20.0% | 10.0% | 20.0% | 15.0% |
| Climbazole | 10.0% | 5.0% | 10.0% | — | — | — |
| Octopirox | — | — | — | 10.0% | 5.0% | 10.0% |
| NMP | — | — | 5.6% | — | — | 5.0% |
| PEG-400 | — | — | — | 12.5% | — | — |
| PG 50-DME | — | — | — | — | — | — |
| SLES | 8.2% | — | 6.0% | — | 5.0% | — |
| CAPB | — | — | 4.0% | — | 5.0% | |
| PG | 34.6% | 24.0% | 18.8% | 18.3% | 18.0% | 20.2% |
| Water | 17.2% | 56.0% | 35.6% | 49.2% | 47.0% | 49.8% |

*Comp = composition

### Example 2:

The process of example 1 is repeated with the antimicrobial active (climbazole, ketoconazole, octopirox, ciclopirox olamine and salicylic acid) and various quaternary ammonium compound (benzalkonium chloride, benzethonium chloride, lauryl pyridinium chloride, tetradecyltrimethylammonium bromide and methyltrioctyl ammonium chloride). The solutions obtained are tested for the activity as per the process given in example 3.

### Example 3: Anti-Microbial Activity Studies:

The antimicrobial efficacy was determined by measuring zone of inhibition using disc diffusion method against E. Coli, Malassezia furfur, Pseudomonas, Aspergillus niger, Staphylococcus and Candida. In this procedure, 10 μ1 of sample (CPC-antimicrobial active combinations obtained in example 1) was added on the filter paper disc and the disc was kept in the microbial culture swabbed on the culture media. The culture plates were incubated at 37° C. for 48 hrs and anti-microbial activity was evaluated by observing an area of no growth around the disc. An area of no growth around the swatch is known as a zone of inhibition. The data obtained against Malassezia furfur is tabulated below:

TABLE 2
Anti-microbial activity data for QAC and antimicrobial active.

| Test conc (in % w/w) | | Zone of inhibition (mm) | | | | | |
|---|---|---|---|---|---|---|---|
| | Antimicrobial | | | | | Ciclopirox | Salicylic |
| QAC | active (AA) | QAC | Climbazole | Ketoconazole | Octopirox | Olamine | Acid |
| 0.25 | 0.25 | — | 36 | 38 | 16 | 12 | 6 |
| 0.25 | 0.5 | — | 36 | 40 | 20 | 16 | 12 |
| 0.25 | 1 | — | 40 | 40 | 20 | 18 | 12 |
| 0.5 | 0.25 | — | 40 | 40 | 20 | 12 | 12 |
| 0.5 | 0.5 | — | 34 | 40 | 24 | 18 | 16 |
| 0.5 | 1 | — | 42 | 40 | 24 | 20 | 20 |
| 1 | 0.25 | — | 40 | 40 | 20 | 16 | 10 |
| 1 | 0.5 | — | 42 | 40 | 20 | 20 | 18 |
| 1 | 1 | — | 40 | 40 | 16 | 20 | 18 |
| 0.25 | — | 8 | — | — | — | — | — |
| 0.5 | — | 16 | — | — | — | — | — |
| 1 | — | 20 | — | — | — | — | — |
| 2 | — | 30 | — | — | — | — | — |
| — | 0.25 | — | 20 | 40 | 10 | 18 | XX |
| — | 0.5 | — | 40 | 40 | 14 | 18 | XX |
| — | 1 | — | 40 | 40 | 18 | 20 | 4 |
| — | 2 | — | 42 | 40 | 20 | 22 | 6 |
| Blank | | | | | | | XX |

XX denotes No Activity and (—) denotes Not Applicable or Not Tested

### Example 4: Synergy Studies

The synergism of the antimicrobial combination of the present invention was determined using the method described by Kull, F. C. et al. in Applied Microbiology, 1961, 9, 538.

The formula to calculate the synergistic index (SI) is

Qa/QA+Qb/QB =SI

Where

QA=concentration of compound A in ppm, acting alone produced an end point

Qa=concentration of compound A in ppm, in the mixture, which produced an end point

QB=concentration of compound B in ppm, acting alone produced an end point

Qb=concentration of compound B in ppm, in the mixture, which produced an end point

Synergism within two compounds is demonstrated when the SI has a value less than 1. The mixtures showed an additive effect if SI is equal to 1 and antagonistic if SI is greater than 1.

The antimicrobial activity results against various microorganisms for QAC-antimicrobial active combinations are tabulated below:

TABLE 2
Synergy data of combinations of CPC with various antimicrobials against different microorganisms

| E. Coli | | | Pseudomonas | | | A. niger | | | S. aureus | | | Candida | | | Malassizia | | |
|---|---|---|---|---|---|---|---|---|---|---|---|---|---|---|---|---|---|
| Qa | Qb | SI | Qa | Qb | SI | Qa | Qb | SI | Qa | Qb | SI | Qa | Qb | SI | Qa | Qb | SI |

Synergy data with Climbazole

| | | | | | | | | | | | | | | | | | |
|---|---|---|---|---|---|---|---|---|---|---|---|---|---|---|---|---|---|
| 5 | 2000 | 0.91 | 250 | 1000 | 0.94 | 10 | 50 | 0.60 | 5 | 1500 | 0.69 | 10 | 100 | 0.50 | 10 | 25 | 0.24 |
| 10 | 500 | 0.26 | 250 | 250 | 0.61 | 10 | 10 | 0.20 | 5 | 1000 | 0.46 | 20 | 100 | 0.60 | 10 | 50 | 0.44 |
| 20 | 2000 | 0.97 | | | | 20 | 50 | 0.70 | 10 | 2000 | 0.91 | 50 | 100 | 0.90 | 10 | 100 | 0.84 |
| 100 | 1000 | 0.84 | | | | 20 | 20 | 0.40 | 10 | 1000 | 0.46 | | | | 25 | 25 | 0.3 |
| 100 | 100 | 0.44 | | | | 20 | 10 | 0.30 | 20 | 2000 | 0.93 | | | | 25 | 50 | 0.5 |
| 100 | 5 | 0.40 | | | | | | | 20 | 1000 | 0.48 | | | | 25 | 100 | 0.9 |
| | | | | | | | | | 50 | 1000 | 0.54 | | | | 50 | 25 | 0.4 |
| | | | | | | | | | 100 | 1000 | 0.64 | | | | 50 | 50 | 0.6 |
| | | | | | | | | | | | | | | | 100 | 25 | 0.6 |
| | | | | | | | | | | | | | | | 100 | 50 | 0.8 |

Synergy data with Octopirox

| | | | | | | | | | | | | | | |
|---|---|---|---|---|---|---|---|---|---|---|---|---|---|---|
| 50 | 500 | 0.70 | 250 | 250 | 0.75 | 50 | 5 | 0.75 | 50 | 500 | 0.70 | 50 | 5 | 0.75 |
| 50 | 250 | 0.45 | 250 | 50 | 0.55 | | | | 50 | 250 | 0.45 | | | |
| 100 | 500 | 0.90 | 250 | 5 | 0.51 | | | | 100 | 500 | 0.90 | | | |
| 100 | 100 | 0.50 | | | | | | | 100 | 50 | 0.45 | | | |
| 100 | 5 | 0.41 | | | | | | | 100 | 5 | 0.41 | | | |

Synergy data with Ciclopirox olamine

| | | | | | | | | | | | | | | |
|---|---|---|---|---|---|---|---|---|---|---|---|---|---|---|
| 0 | 2000 | 1 | 0 | 2000 | 1 | 0 | 2000 | 1.00 | 0 | 2000 | 1 | 0 | 2000 | 1.00 |
| | | | 250 | 500 | 0.75 | 5 | 1000 | 0.55 | 50 | 1000 | 0.7 | 50 | 500 | 0.75 |
| | | | 250 | 50 | 0.52 | 5 | 500 | 0.30 | 100 | 1000 | 0.9 | 50 | 100 | 0.55 |
| | | | | | | | | | 100 | 100 | 0.45 | 50 | 5 | 0.5 |
| | | | | | | | | | 100 | 5 | 0.4 | | | |

Synergy data with Ketoconazole

| | | | | | | | | | | | | | | |
|---|---|---|---|---|---|---|---|---|---|---|---|---|---|---|
| 5 | 2000 | 0.91 | 5 | 2000 | 0.90 | 50 | 250 | 0.70 | 50 | 5 | 0.75 | 25 | 2.5 | 0.3 |
| 5 | 1500 | 0.69 | 10 | 2000 | 0.91 | 50 | 100 | 0.40 | | | | 25 | 10 | 0.9 |
| 10 | 2000 | 0.93 | 20 | 1500 | 0.71 | 100 | 250 | 0.90 | | | | 100 | 2.5 | 0.6 |
| 10 | 1500 | 0.71 | 20 | 500 | 0.26 | 100 | 100 | 0.60 | | | | 100 | 5 | 0.8 |
| 20 | 2000 | 0.97 | 20 | 250 | 0.15 | | | | | | | | | |
| 20 | 1000 | 0.52 | 50 | 1500 | 0.77 | | | | | | | | | |
| 100 | 1000 | 0.84 | 50 | 250 | 0.21 | | | | | | | | | |
| 100 | 250 | 0.51 | 50 | 5 | 0.10 | | | | | | | | | |
| 100 | 100 | 0.44 | 100 | 1500 | 0.87 | | | | | | | | | |
| | | | 100 | 100 | 0.24 | | | | | | | | | |
| | | | 250 | 1000 | 0.94 | | | | | | | | | |

Synergy data with Salicylic acid

| | | | | | | | | | | | |
|---|---|---|---|---|---|---|---|---|---|---|---|
| 50 | 2000 | 0.83 | 10 | 1000 | 0.52 | 5 | 3000 | 0.96 | 10 | 2000 | 0.77 |
| 100 | 1000 | 0.71 | 50 | 1000 | 0.60 | 10 | 3000 | 0.98 | | | |
| 100 | 100 | 0.43 | | | | 50 | 2000 | 0.83 | | | |
| 100 | 5 | 0.40 | | | | | | | | | |

We claim:

1. A synergistic antimicrobial composition comprising quaternary ammonium compound and antimicrobial active.

2. The synergistic antimicrobial composition as claimed in claim 1, wherein quaternary ammonium compound is selected from the group comprising methyltrioctyl ammonium halides, cetyltrimethyl ammonium halides, decyltrimethyl ammonium halides, didecyldimethyl ammonium halides, trimethyltetradecyl ammonium halides, methyl pyridinium halides, ethyl pyridinium halides, cetrimonium halides, dodecyl (lauryl) pyridinium halides, tetradecyl (myristyl) pyridinium halides, hexadecyl (cetyl) pyridinium halides, octadecyl(stearyl) pyridinium halides, alkylbenzyldimethyl ammonium halides, benzalkonium halides or benzalkonium saccharinates with alkyl chain lengths of C₈-C₁₈ ,and combinations thereof.

3. The synergistic antimicrobial composition as claimed in claim 1, wherein the antimicrobial active is selected from the group comprising abafungin, albaconazole, bifonazole, butoconazole, clotrimazole, climbazole, econazole, efinaconazole, epoxiconazole, fluconazole, fenticonazole, isavuconazole, itraconazole, isoconazole, ketoconazole, luliconazole, miconazole, omoconazole, oxiconazole, posaconazole, propiconazole, ravuconazole, sertaconazole, sulconazole, spectrazole, tioconazole, terconazole, tolyltriazole, voriconazole, ciclopirox olamine, octopirox, selenium sulfide, sulfur, coal tar, salicylic acid, benzotriazole, 2-mercaptobenzothiazole (MBT), lactic acid, pyruvic acid, urea, benzoyl peroxide, N-acetylcysteine, retinoids, tretinoin, retinoic acid, retinol and retinol palmitate, isotretinoin-13-cis-retinoic acid, tetracycline, erythromycin, minocycline, clindamycin, tolnaftate, terbinafine, methyl-isothiazolinone, chloromethyl-isothiazolinone, benzisothiazolinone, octyl-isothiazolinone, dichlorooctyl-isothiazolinone, butyl-benzisothiazolinone, iodopropynyl butylcarbamate and combinations thereof.

4. The synergistic antimicrobial composition as claimed in claim 1, wherein the quaternary ammonium compound is selected from the group comprising methyltrioctyl ammonium chloride, methyltrioctyl ammonium bromide, cetyl trimethyl ammonium chloride, cetyl trimethyl ammonium bromide, decyltrimethyl ammonium chloride, trimethyltetradecyl ammonium bromide, dodecyl (lauryl) pyridinium chloride, tetradecyl (myristyl) pyridinium halides, benzethonium chloride, cetrimonium chloride, cetrimonium bromide, benzalkonium chloride, benzalkonium bromide, benzalkonium saccharinate, cetylpyridinium chloride, cetylpyridinium bromide and octadecylpyridinium chloride and combinations thereof.

5. The synergistic antimicrobial composition as claimed in claim 1, wherein the antimicrobial active is selected from the group comprising, clotrimazole, climbazole, fluconazole, ketoconazole, ciclopirox olamine, octopirox, selenium sulfide, sulfur, salicylic acid and combinations thereof.

6. A synergistic antimicrobial composition comprising cetylpyridinium chloride and an antimicrobial active selected from climbazole, ketoconazole, ciclopirox olamine, octopirox, salicylic acid and combinations thereof.

7. The synergistic antimicrobial composition as claimed in claim 1 or 6, wherein the ratio of quaternary ammonium compound to the antimicrobial active is 1:5 to 5:1.

8. The synergistic antimicrobial composition as claimed in claim 1 or 6, wherein the said composition is transparent or opaque.

9. The synergistic antimicrobial composition as claimed in claim 1 or 6, for use in the manufacture of antidandruff, anti-acne, anti-wart, anti-fungal, anti-eczema, anti-psoriasis, anti-athlete's foot, anti-ringworm, anti-pruritic, anti-candidiasis, anti-crack, anti-dermatitis, anti-tinea, anti-vitiligo, would healing and dry skin formulations.

10. A process for preparing the synergistic antimicrobial composition as claimed in claim 1 or 6, said process comprising:
(a) preparing a solution of antimicrobial active in suitable solvent,
(b) preparing a solution of quaternary ammonium compound in suitable solvent,
(c) adding quaternary ammonium compound solution to antimicrobial active solution,
(d) adding aqueous mixture of rheology modifier, pH regulator, preservative optionally.

11. A personal care, cosmetic, pharmaceutical, home care, hospital care, laundry care and industrial formulation comprising the synergistic antimicrobial composition as claimed in claim 1 or 6.

12. A method for inhibiting microbial growth on a surface comprising applying to the said surface the synergistic antimicrobial composition as claimed in claim 1 or 6.

13. Use of quaternary ammonium compounds for inhibiting the growth of Malassezia spp.

14. Use of cetylpyridinium chloride in an amount of 0.05 -10% for inhibiting the growth of Malassezia spp.
